(19) United States
(12) Patent Application Publication
(10) Pub. No.: US 2019/0150767 A1
(43) Pub. Date: May 23, 2019

(54) WEARABLE HEALTHCARE DEVICE

(71) Applicant: PHYSIO-CONTROL, INC., REDMOND, WA (US)

(72) Inventors: Karen E. McEwen, Kenmore, WA (US); Erick Michael Roane, Kirkland, WA (US); Cameron Pollock, Redmond, WA (US); Kyle Cote, Redmond, WA (US)

(21) Appl. No.: 16/255,586

(22) Filed: Jan. 23, 2019

Related U.S. Application Data
(62) Division of application No. 15/311,180, filed on Nov. 14, 2016.
(60) Provisional application No. 62/026,517, filed on Jul. 18, 2014.
(60) Provisional application No. 61/992,178, filed on May 12, 2014.

(51) Int. Cl. A61B 5/024; A61B 5/00; A61B 5/021; A61B 5/0205; A61H 31/00; A61N 1/39; A61B 5/11; A61B 5/145; G09B 19/00; A61B 5/0432

(52) U.S. Cl. CPC A61B 5/02438; A61B 5/02455; A61B 5/021; A61B 5/742; A61B 5/0205; A61H 31/005; A61H 31/007; A61N 1/3993; A61B 5/1112; A61N 1/3975; A61B 5/6832; A61B 5/6828; A61B 5/6824; A61B 5/6822; A61B 5/14542; G09B 19/003; A61B 5/11; A61B 5/0432; A61H 2201/5084; A61B 5/6831; A61H 2201/5071; A61H 2201/5058; A61H 2201/5048; A61H 2201/5043; A61H 2201/501; A61H 2201/165; A61H 2201/5097; A61H 2230/06; A61H 2230/10; A61H 2230/207; A61B 5/0004

(57) ABSTRACT

Technologies and implementations for wearable healthcare devices are generally disclosed.

### RELATED APPLICATION

This application claims benefit of priority to U.S. Provisional Patent Application Ser. No. 61/992,178, filed on May 12, 2014, titled Smart Patient Therapy Information Tag and U.S. Provisional Patent Application Ser. No. 62/026,517, filed on Jul. 18, 2014, titled LED Optical Indicators for Pulse Sensor and Metronome Synchronization. Both U.S. Provisional Patent Applications, 61/992,178 and 62/026,517, are incorporated herein by reference in their entirety.

### BACKGROUND

Unless otherwise indicated herein, the approaches described in this section are not prior art to the claims in this application and are not admitted to be prior art by inclusion in this section.

As wearable devices become more “smart” and become more packed with capabilities, people are finding various ways to use wearable devices to help their lives. Uses of wearable devices may include a wide range of areas. One example area, where wearable devices may be used, may be in the health related area. For example, wearable devices may help facilitate assistance with healthcare related issues.

### SUMMARY

The present disclosure describes example methods, apparatus, and systems related to wearable healthcare device. An example apparatus may include a wearable healthcare apparatus having a processor, a pulse sensor communicatively coupled to the processor. The pulse sensor may be configured to sense an indication of a pulse of the wearer of the wearable healthcare apparatus. The example apparatus may also include a visual indicator device. The visual indicator device may be configured to synchronously visually indicate the pulse of the wearer of the wearable healthcare apparatus.

Another example apparatus may include a wearable healthcare apparatus having a processor, a wearable healthcare utilization module (WHUM) communicatively coupled to the processor. The example apparatus may also include a storage medium communicatively coupled to the WHUM. The example apparatus may include a transmit/receiver (TX/RX) device, a global positioning system (GPS) device, a display device, and a pulse sensing device. Additionally, the example apparatus may include a power supply configured to provide energy to at least the wearable healthcare apparatus and also configured to provide a predetermined energy to facilitate defibrillation of a human heart.

Another example apparatus may include a wearable healthcare apparatus configured to facilitate cardiopulmonary resuscitation (CPR) training.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

In the drawings:

FIG. 1 illustrates a block diagram of a wearable healthcare device, in accordance with various embodiments;

FIG. 2 illustrates a block diagram of a wearable healthcare device, in accordance with various further embodiments;

FIG. 3 illustrates a block diagram of a wearable healthcare device, in accordance with various further embodiments;

FIG. 4 is a block diagram illustrating components of a wearable device, in accordance with various embodiments;

FIG. 5 illustrate an operational flow for a wearable healthcare device, arranged in accordance with at least some embodiments described herein;

FIG. 6 illustrate an operational flow for an emergency apparatus capable of indicating its location, arranged in accordance with at least some embodiments; and

FIG. 7 is a block diagram illustrating an example computing device 700, such as might be embodied by a person skilled in the art, which is arranged in accordance with at least some embodiments of the present disclosure.

### DETAILED DESCRIPTION

The following description sets forth various examples along with specific details to provide a thorough understanding of claimed subject matter. It will be understood by those skilled in the art, however, that claimed subject matter may be practiced without some or more of the specific details disclosed herein. Further, in some circumstances, well-known methods, procedures, systems, components and/or circuits have not been described in detail in order to avoid unnecessarily obscuring claimed subject matter.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

This disclosure is drawn, inter alia, to methods, apparatus, and systems related to a wearable healthcare device. Such a wearable healthcare device may include various electronic devices. An example of a wearable healthcare device may include a display, a processor, and/or a pulse sensor among various other electronic devices.

Information about a person's health may be available through various methodologies. However, some of these methodologies may include using sophisticated devices such as, but not limited to, smart phones. Smart phones may be capable of providing a great deal of healthcare related information. However, much of this healthcare related information might not be necessarily useful to an average person. Additionally, smart phones may be used in conjunction with wearable devices, which may be closer to a person's body to sense and/or detect healthcare related information. As will be described in the present disclosure, wearable devices may be configured to provide information and functionality, which may be considered to be useful for a person's healthcare. Before turning to the Figures, a number of non-limiting examples will be described.

Continuing with the non-limiting example of a wearable device related to healthcare, a wearable healthcare device (hereon out, “WD”) may be worn on various parts and/or surfaces of a person. For example, a WD may be worn on a wrist. The WD may include at least a processor, a display, and a sensor.

In one example, a WD may be configured to provide simple, yet useful, information such as, but not limited to, a pulse of a wearer. For example, a WD may be configured to be worn on a wrist of a user. As the WD is worn, a pulse sensor may detect the pulse of the wearer. A visual indication of the pulse of the wearer may be displayed, where the visual indication may be synchronized with the pulse (e.g., a blinking light of some kind). Continuing with the non-limiting example of the WD capable of providing a visual indication of wearer's pulse, an example scenario may include where a person may be wearing the WD. The person may experience some event related to the heart such as, but not limited to, arrhythmia. Some arrhythmias may lead to a condition known as sudden cardiac arrest (SCA). Example conditions of SCA may include atrial fibrillation (AF) and/or ventricular fibrillation (VF), where the heart muscles may basically quiver or beat irregularly which may result in blood not being pumped properly. The WD may be configured to detect such an event (e.g., the processor may detect the irregularity with the pulse as the WD continually senses the wear's pulse). Because of such a condition, the WD may be configured to start blinking, and the blinking may be synchronized with the pulse. The irregular blinking light may facilitate indication of the fibrillation event. The blinking light may also provide a potential rescuer an indication of pulse.

In another example, the WD configured to provide the visual indication of the pulse may also be configured to provide a timing indicator for the potential rescuer. For example, the visual indicator for the pulse may provide a metronome function for the rescuer during cardiopulmonary resuscitation (CPR) being performed on the WD wearer. The metronome function may facilitate assistance in keeping an appropriate rhythm for the CPR (e.g., chest compressions). The visual indicator for the metronome function may be the same and/or different from the visual indicator for the pulse.

In another example, the WD configured to provide the visual indication of the pulse may also include a storage device. Under control of a processor, the storage device may store information regarding the wearer. For example, the storage device may have data regarding the pulse of the wearer for a predetermined period of time. Perhaps the wearer of the WD may have a condition, where the wearer may be at risk of some circulatory and/or heart event. The data may provide useful information to a healthcare professional and/or to the wearer. Additionally, the WD may include communicative capabilities such as, but not limited to, wired and/or wireless signal capabilities. The communicative capabilities may facilitate providing various data related to the wearer to a healthcare professional and/or to the wearer. In the example of a healthcare professional, the healthcare professional may use the data to determine the health of the wearer. Additionally, the communicative capabilities may be utilized as an alert for various healthcare and/or emergency personnel.

In continuing with the non-limiting example of a person having a health condition, where the wearer may be at risk of some circulator and/or heart event, a WD may include a display having various data to be displayed and/or transmitted to a potential rescuer and/or emergency personnel. For example, the WD may include a display, a motion sensor, and/or a global positioning (GPS) device. An example scenario may include, where a person wearing the WD suffers a heart related event, such as but not limited to, VF. The WD may detect the event via the pulse sensor and/or the motion sensor (e.g., the pulse of the wearer indicates some circulatory and/or heart related event and/or the motion sensor detects a fall). Once the health related event is detected, the WD may transmit its location (e.g., via a wireless signal medium) to a healthcare and/or emergency facility. On the display of WD, various information such as, but not limited to, pulse, name, address, geographic location of the WD (i.e., location of the wearer), etc., and/or any combination thereof may be displayed.

In addition to communicating with healthcare and/or emergency personnel, the WD may include a vibration device and/or a speaker to facilitate alerting the wearer of the WD that a potential circulatory and/or heart even may be imminent or occurring.

In addition to facilitating detection of a health related event, the motion sensor (e.g., an accelerometer) may facilitate aiding a potential rescuer in performing various life saving activities such as, but not limited to, CPR. For example, in this scenario, a potential rescuer may be wearing a WD having, among various other functionalities, a motion sensor. The motion sensor may facilitate assistance with performing of the CPR. Assistance may include frequency and/or depth of chest compressions based, at least in part, on the motion detected by the motion sensor. In an example, where both the potential rescuer and the person experiencing the event, may be wearing a WD each respectively, the two WDs (e.g., potential rescuer and the person experiencing the event) may include wireless communication capabilities such as, but not limited to, radio-frequency identification (RFID), Bluetooth, near field communication (NFC), etc., and/or any combination thereof. In the example of RFID, when the two WDs are within the wireless capabilities of each other, the two WDs may detect each other and data may be wirelessly communicated between the two WDs (e.g., healthcare information from the person experiencing the event to the potential rescuer).

Continuing with the non-limiting example of performing CPR, a WD may be configured to facilitate CPR training. For example, a CPR trainee may wear one WD having at least some of the various functionalities, as previously described, while another WD may be worn by a CPR trainer. In this example scenario, as the trainee performs CPR (either on a mannequin or on a person), the WD having the CPR training capabilities worn by the CPR trainee may be configured to provide various guidance. For example, the WD worn by the CPR trainee may provide guidance by emitting an audible and/or a visual cue when to apply chest compression. Additionally, a WD on the CPR trainer may be in communication with the WD on the CPR trainee. Various data related to the performance of the CPR trainee may be communicated to the WD on the CPR trainer. As part of the communication, the CPR trainer may be capable of adjusting assistance provided by the WD on the CPR trainee. For example, if the data received by the WD on the CPR trainer from the WD on the CPR trainee seems to indicate that the CPR trainee is not compressing deep enough, the CPR trainer may communicate, via the WD on the CPR trainer, an indication of the performance. One example communication may be in the form of causing the WD on the CPR trainer to indicate an audible command such as, but not limited to, “Please increase compression depth”. In another example, the display device on the WD on the CPR trainee may indicate textually by displaying a message such as, but not limited to, “Please increase compression depth”. If follows that any type of command/communication may be facilitated.

In a further example, a person experiencing a healthcare related event may have a WD, a potential rescuer may have a WD, and a third person may have a WD. The three WDs may be capable of communicating with each other. An example scenario may be where a person may be experiencing a healthcare related event such as, but not limited to, VF. As previously described, the WD on the person experiencing the event may have indicated the event and/or communicated the event to an emergency personnel. The potential rescuer to first arrive to provide assistance to the person experiencing the event may not necessarily be emergency personnel. However, because the potential rescuer may be the first person on the scene, the potential rescuer may start to perform or be performing life saving activity such as, but not limited to, CPR. A third person (e.g., emergency personnel) may arrive after the potential rescuer. In this example scenario, the WD on the person experiencing the event, the WD on the potential rescuer, and the WD on the emergency personnel may be capable of communicating with each other to coordinate the required assistance for the person experiencing the event. For example, the WD on the person experiencing the event may communicate a wide variety of information to the WD on the potential rescuer and/or the emergency personnel regarding the person experiencing the event such as, but not limited to, pulse, name, address, height, age, weight, medical history, allergy information, preexisting medical conditions, etc., and/or any combination thereof. At least some of the communicated information may be useful for the potential rescuer and/or the emergency personnel. For example, the potential rescuer may not be CPR trained or have limited CPR training. The information communicated from the WD on the person experiencing the event may help the emergency personnel in assessing the effectiveness of the CPR. The emergency personnel may, using the WD on the emergency personnel, cause the WD on the potential rescuer to provide guidance on the life saving activity such as, but not limited to, an audio indication of “Please increase depth of compressions”, “Please increase the rate of compression”, or a visual indication to be followed by the potential rescuer for chest compressions (e.g., a periodic blinking light providing a metronome type of indication).

In the above example scenario, the potential rescuer and/or the emergency personnel may have access to an automated external defibrillator (AED) type device. In another example, at least one or more of the WDs may be capable of communicating with the AED device. Communication with the AED device may facilitate location of an AED and/or facilitating providing data to the AED device. Additionally, a WD may include data as to location of an AED device based, at least in part, on a current location of the WD. As previously described, the location of the WD may be determined with the assistance of a GPS device included in the WD.

Once the person experiencing the event may have been transported to a medical facility (hereon out, “patient”), the WD on the patient may be utilized as a patient information band. For example, information regarding the patient may be stored and displayed on the display device included in the WD for medical personnel. In another example, the information regarding the patient, including information regarding the activities of the potential rescuer, may be communicated to a computer at the medical facility or to a network communicatively coupled to the medical facility.

Continuing with the non-limiting example of the patient being located at the medical facility, the WD worn by the patient may be capable of facilitating tracking of the patient within a geographic area such as, but not limited to, the medical facility. For example, the patient may move about the medical facility, and the movement of the patient may be tracked as the WD worn by the patient may communicate its location with a network such as, but not limited to, the network of the medical facility. Additionally, the WD worn by the patient may be capable of periodically updating information about the patient. Information such as, but not limited to, the health condition of the patient, may be updated periodically to the network of the medical facility, where the information may be accessible by medical personnel. In one example scenario, a patient may be wearing a WD and moving about the medical facility. As the WD continually monitors the wearer (i.e., the patient), the WD may detect a healthcare related event such as, but not limited to, VF (hereon out, medical emergency). The WD may be capable of providing an indication of the medical emergency to the wearer, to the surroundings (e.g., people who may be nearby to potentially provide assistance), and/or to emergency personnel (e.g., medical personnel within the medical facility). As previously described, the indication of the medical emergency by the WD may include an audio indication such as, but not limited to, a siren and/or a voice and/or a visual indication such as, but not limited to, a blinking light. The blinking light may include a single color or various colors to facilitate the indication of the medical emergency. Additionally, the WD may be capable of communicating the medical emergency via an electronic communication medium such as, but not limited to, a wireless communication medium. The communication may be to a wireless communication medium network such as, but not limited to, a wireless network of the medical facility.

The responding medical personnel may also be wearing a WD, which would facilitate at least some of the non-limiting scenarios previously described such as, but not limited to, the CPR trainee/trainer scenario. Additionally, it may be appreciated that the various scenarios may be expanded to include a wide range of procedures, where guidance may be utilized, such as, but not limited to, administering drugs, measuring blood pressure, etc., and/or any combination thereof. For example, the WD may include a peripheral capillary oxygen saturation device (e.g., SpO2), which may facilitate detection of oxygen saturation levels in the wearer's blood. In another example, the WD may be worn around a person's arm, where strap(s) holding the WD on the person's arm may be capable of being actuated to tighten and/or loosen under the control of the WD to facilitate measuring blood pressure of the person wearing the WD. For example, the WD would be capable of detecting the pulse of the wearer and as the strap holding the WD is tightened and loosened around arm, the blood pressure of the person wearing the WD may be measured.

In yet another example, a WD may be capable of providing a potentially life saving procedure such as, but not limited to, defibrillation of a heart. Continuing with the non-limiting example of the medical related issues, heart related issues have become prevalent throughout many parts of the World. For example, coronary heart disease may lead to issues related to the heart such as, but not limited to, arrhythmia as previously described. In order to treat a heart in a condition of VF, the heart may need to be defibrillated by the application of an electrical signal (e.g., an electric shock). In this example, a WD may include defibrillator device capabilities and may facilitate administration of an electrical shock to the heart, thereby defibrillating the heart undergoing VF.

A challenge with defibrillation may be that the electrical shock should be administered very soon after the onset of VF. In order to facilitate administration of electrical shock soon after the onset of VF, alerting emergency personnel and/or bystanders of the need for treatment of VF may be necessary. There may be a variety of ways to alert emergency personnel and/or bystanders, such as, but not limited to, examples involving WDs as previously described (e.g., if the WD detects the onset of VF, the WD may be capable of transmitting an electronic signal to alert emergency personnel and/or bystanders). However, a WD worn by a person undergoing VF may facilitate faster administration of the electrical shock.

In the example of a WD having defibrillator capabilities, the WD may include retractable wires having electrodes coupled to the retractable wires. Commonly, defibrillation may include two electrodes placed on the person undergoing VF. Accordingly, the WD may include two retractable wires. The wires may be retractable to facilitate being worn by a wearer. For example, the WD may be worn on a wrist, around a neck, a leg, around the body, and any other part of a person. Additionally, a power supply may be electrically coupled to the WD, where the power supply may be capable of providing enough energy to the WD to facilitate defibrillation. An example energy level may be approximately 100 Joules to 350 Joules based, at least in part, on the age, gender, weight, age, etc., and/or any combination thereof of the person to receive the electric shock.

In a non-limiting scenario, a person wearing a WD may experience a healthcare related event such as, VF. The WD may facilitate indication of the VF in a variety of manners such as, but not limited to, the previous examples described. The person experiencing the VF (hereon out, “VF person”) may be incapacitated. A potential rescuer may arrive on the scene and may start some form of potentially life saving procedure such as, but not limited to, CPR. The potential rescuer may notice that the VF person is wearing a WD having capabilities of a defibrillator device. As part of the potentially life save procedure, the potential rescuer may pull out the electrodes from the WD and place the electrodes on the VF person. The locations of the electrodes may be guided by a display included on the WD and/or by audio cues from the WD. For example, the WD may have detected that the electrodes have been pulled out from its retracted position causing the WD to initiate defibrillator procedures including visual and/or audio commands. The potential rescuer may activate the WD for defibrillating the VF person following various commands/cues such as those that may be displayed/heard from the WD.

Alternatively, the potential rescuer may be the one wearing a WD having capabilities of a defibrillator device. The potential rescuer may activate the defibrillator capabilities of their own WD to facilitate defibrillation of the VF person. As previously described, the potential rescuer may be trained to use the WD by a trained emergency personnel who may have arrived on the scene after the potential rescuer.

As may be appreciated by the disclosure so far, the disclosed examples of WDs may be capable of a wide variety of uses/applications, and accordingly, this and further disclosed subject matter fall within the scope of the claimed subject matter.

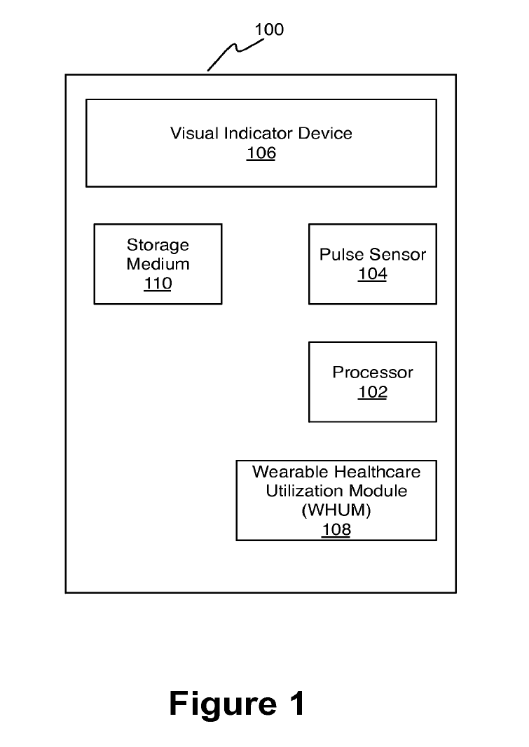

Turning now to the figures, FIG. 1 illustrates a block diagram of a wearable healthcare device, in accordance with various embodiments. In FIG. 1, a wearable healthcare device (hereon out, WD) 100 may include a processor 102, a pulse sensor 104, a visual indicator device 106, a wearable healthcare utilization module (WHUM) 108, and a storage medium. The WHUM 108 may be communicatively coupled to the processor 102, and under the control of the processor 102, the WD 100 may have at least the functionality described herein.

In one example, the visual indicator device 106 may include one or more LEDs, which may be configured to blink synchronously with a pulse of the wearer. The pulse sensor 104 may detect the pulse of the wearer and provide the pulse information to the processor 102 to facilitate blinking of the visual indicator device 106. The LEDs may blink in a manner to indicate information regarding the pulse such as, but not limited to, intensity of the detected pulse (e.g., strength of the pulse). Additionally, the LEDs may be of different colors to provide information regarding the pulse. For example, green LEDs to indicate that the pulse is within normal boundaries, while yellow to red LEDs to indicate that the pulse may be outside the normal boundaries with yellow being just outside and red being far outside the normal boundaries.

In another example, the visual indicator device 106 may be a liquid crystal display (LCD) type device to facilitate indication (e.g., display) of information regarding the wearer such as, but not limited to, pulse information, name, address, location information, information previously described with respect to the various examples disclosed, etc., and/or any combination thereof.

In yet another example, the storage medium 110 may be configured to store electrocardiography (ECG) data of a wearer. As previously described, the ECG data, among other information, may be electrically communicated to a network and/or to other devices such as, but not limited to, a computer, a server, another WD, a medical device (e.g., AED device), etc., and/or any combination thereof.

In some examples, the WD 100 may be configured to be wearable on various parts of a person such as, but not limited to, a wrist, a leg, around a neck (e.g., a necklace), a surface of clothing (e.g., as a broach/pin), on the surface of the skin (e.g., with the application of an adhesive), as part of a covering on a medical apparatus (e.g., a covering wrapping a stethoscope), as part of headphones/ear buds, headbands, hats, helmets, anklets, ankle band, earrings, etc., and/or any combination thereof. Accordingly, the term wearable, as described, encompasses a wide range of wearing.

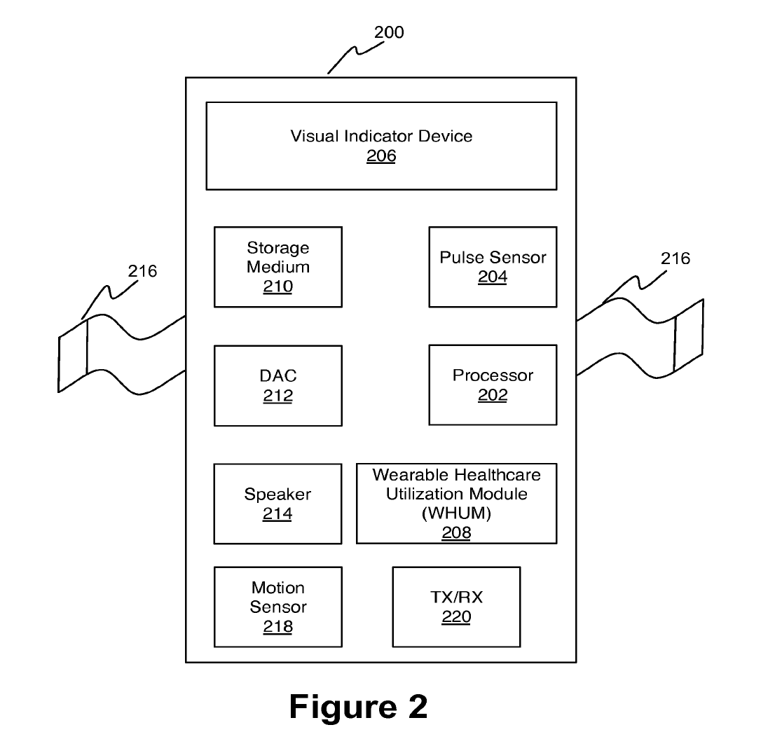

FIG. 2 illustrates a block diagram of a wearable healthcare device, in accordance with various further embodiments. In FIG. 2, a WD 200 may include similar function blocks as shown in the WD 100 (shown in FIG. 1). The WD 200 may include a processor 202, a pulse sensor 204, a visual indicator device 206, a wearable healthcare utilization module (WHUM) 208, and a storage medium 210. In one example, the WD 200 may include a digital to analog converter (DAC) 212 communicatively coupled to the processor 202. A speaker 214 may be communicatively coupled to the DAC 212. As previously described, the speaker 214 may facilitate audio indication of various information, cues, indications, etc., and/or any combination thereof.

In another example, the WD 200 may include a band 216. The band 216 may be configured to go around an arm of the wearer. The band 216 may be capable of tightening and loosening controlled by the processor 202. The band 216 may tighten and loosen around the arm of the wearer by inflating and deflating. Accordingly, the band 216 may be an inflatable band. The band 216 in conjunction with the pulse sensor 204 may facilitate measuring a blood pressure of the wearer.

In another example, the WD 200 may include a motion sensor 218. The motion sensor 218 may be communicatively coupled to the processor 202. The motion sensor 218 may facilitate sensing motion related to the wearer of the WD. Some examples of the sensed motion may include, but not limited to, at least some of the examples previously described such as, but not limited to, motion related to CPR (e.g., range of motion related to CPR). In another example, the WD 200 may include a transmit and/or receive module (TX/RX) 220 communicatively coupled to the processor 202. The TX/RX module 220 may facilitate electrical communication of various information of the wearer such as, pulse, blood pressure, oxygen level in the blood, location of the WD 200, etc., and/or any combination thereof.

The TX/RX module 220 may be capable of using a variety electrical communication mediums including wired and wireless communication mediums such as, but not limited to, near field communication (NFC) type electronic communication medium, radio-frequency identification (RFID) type of wireless electronic communication medium, Bluetooth wireless electronic communication medium, wireless local area network (WLAN) type wireless electronic communication medium (e.g., IEEE 802.11 and its variations and/or extensions), and so forth. Accordingly, the claimed subject matter is not limited in these respects. The motion sensor 218 may include a wide range of sensors such as, but not limited to, microelectromechanical system (MEMS) including accelerometers, gyroscopes, digital compasses, inertial modules, pressure sensors, humidity sensors, microphones, speakers, smart sensors, sensor hubs, UV index sensors, temperature sensor, touch sensor, etc., and/or any combination thereof.

As may be appreciated, the disclosed subject matter may utilize various sensors (e.g., pulse sensor 104/204 and/or motion sensor 218. Accordingly, a wide range of sensors may be included such as, but not limited to, sensors, which may be capable of being utilized to detect various biomarkers on a human body. For example, along with at least some of the components shown in the WDs (shown in FIGS. 1 and 2), a sensor capable of detecting biomarkers, which may be included in human sweat, such as, but not limited to, electrolytes, sodium, lactates, proteins, etc., and/or any combination thereof. Additionally, a MEMS type device such as, but not limited to, a total analysis system (μTAS), which may be associated with a lab-on-a-chip (LOC) type system, may be included as a sensor in the WDs. For example, these microfluidic type devices may be capable of obtaining a wide variety of information regarding a wearer such as, but not limited to, a wide range of molecular information. Some examples of molecular information may include, but not limited to, deoxyribonucleic acid (DNA) information, pH level information, cytometry information, chemical gradient information, etc., and/or any combination thereof. The information of the wearer may be communicated to various destinations for assistance in determining the health of the wearer.

In one example, a sensor may be capable of detecting various biomarkers of the wearer, where certain levels of biomarkers may be an indication of a health related condition/event such as, but not limited to, a elevated level of stress, fatigue, sugar levels, sodium levels, etc., and/or any combination thereof. Responsive to the detected levels of biomarkers, the WD 100/200 may be configured to be capable of providing a visual and/or audio indication such as, but not limited to, blinking light, a display showing the levels, etc., and/or any combination thereof.

It should be appreciated that the block diagrams of the WD 100 and the WD shown in FIGS. 1 and 2 may be shown with various functional blocks such as, but not limited to the processor 102/202, pulse sensor 104/204, visual indicator device 106/206, WHUM 108/208, storage medium 110/210, band 216, motion sensor 218, and TX/RX module 220. However, the WD 100 and WD 200 may be shown with and/or without these and other various functional blocks without departing from the spirit and scope of the disclosed subject matter. As previously described, the WD 100 and WD 200 may be wearable in a wide variety of manners such as, but not limited to, around a neck as a necklace, around a leg, around an arm, on skin, etc., and/or any combination thereof.

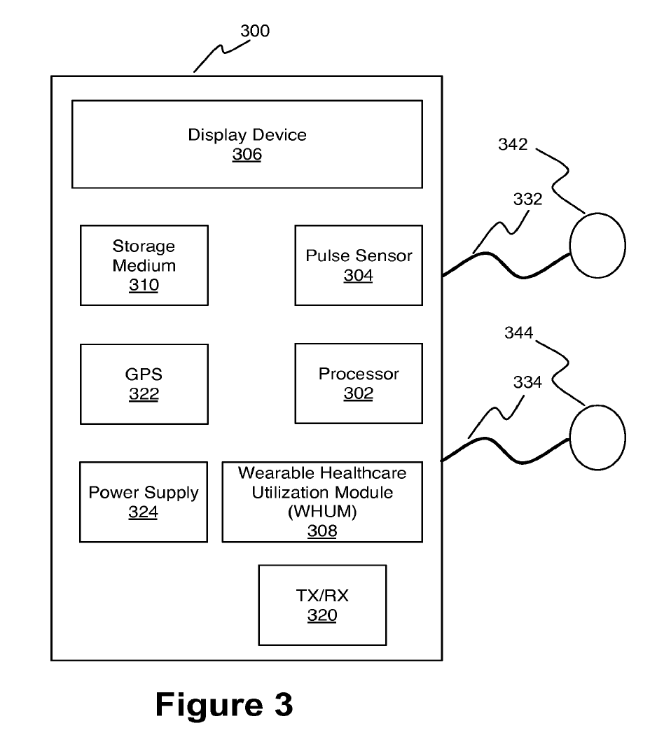

FIG. 3 illustrates a block diagram of a wearable healthcare device, in accordance with various further embodiments. In FIG. 3, a WD 300 may include similar function blocks as shown in the WD 100 (shown in FIG. 1) and the WD 200 (shown in FIG. 2). The WD 300 may include a processor 302, a pulse sensing device 304, a display device 306, a wearable healthcare utilization module (WHUM) 308, a TX/RX module 320, and a storage medium 310. In one example, the WD 300 may include a global positioning system (GPS) device 322. In another example, the WD 300 may include a power supply 324. In the example shown in FIG. 3, the WD 300 may additionally include a first wire 332 and a second wire 334. The first and second wire 332 and 334 may be electrically coupled to the WD 300. Additionally, in one example, the first and second wire 332 and 334 may be capable of being retracted into the WD 300 and being capable of being pulled out of the WD 300. In FIG. 3, the first wire 332 may have a first electrode 342 electrically coupled to an end of the first wire 332, and the second wire 334 may have a second electrode 344 electrically coupled to an end of the second wire 334. In the example shown in FIG. 3, the power supply 324 may be configured to provide a predetermined energy to facilitate defibrillation of a human heart. Accordingly, the WD 300 may be capable of being utilize as an AED device.

The first and the second wire 332 and 334 may be a wide variety of wires capable of supporting energy levels for supporting AED device capabilities. The first and second electrodes 342 and 344 may be a wide variety of electrodes capable of electrically transferring energy to a person such as, but not limited to, electrodes, which may be used in conjunction with AEDs. The WD 300 may help facilitate defibrillation of the person's heart undergoing some healthcare related event such as, but not limited to, a VF event. It follows that the power supply 324 may be wide variety of power supplies capable of providing predetermined energy levels to help facilitate defibrillation of a person experiencing VF.

The GPS device 322 may be capable of receiving location information of the WD 300 from a GPS network (e.g., satellite system). As previously described, the location information may be stored in the storage medium 310. In one example, the location information may be transmitted via the TX/RX module 320 to a network to help facilitate to location of the WD 300.

It should be appreciated that the WD 300 may include sensors such as, but not limited to, the sensors previously described with respect to FIGS. 1-2.

It should be appreciated that the block diagrams of the WD 100, the WD 200, and WD 300 shown in FIGS. 1, 2, and 3 may be shown with various functional blocks being located within the bounds of the WDs. However, these and any other functional block shown and/or not shown may be within the bound and/or outside the bounds of the WDs without departing from the scope and spirit of the claimed subject matter. For example, the power supply 324 may be included in the WD 300 or may be electrically coupled to the WD 300 externally. Accordingly, the claimed subject matter is not limited in these respects.

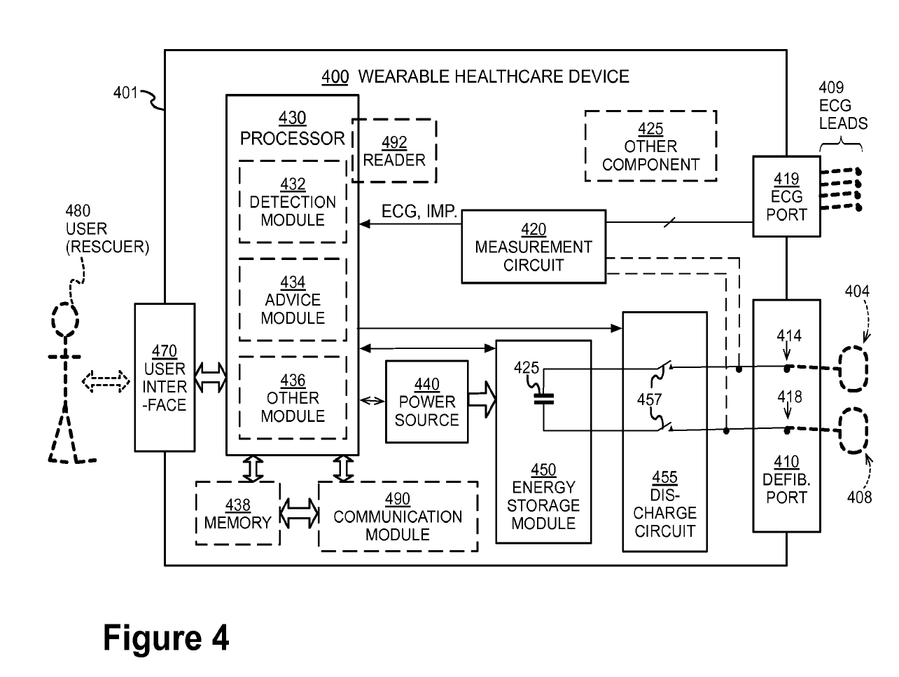

FIG. 4 is a block diagram illustrating components of a wearable device, in accordance with various embodiments. In FIG. 4, a WD 400 may be configured to include the capabilities of an AED device such as, but not limited to, the WD 300 described with respect to FIG. 3, and these components may be, for example, the WD 300. Additionally, the components of FIG. 3 may be provided in a wearable housing 401 (hereon out, WH).

The WD 400 may be intended for use by a user 480 (e.g., a potential rescuer). The WD 400 may typically include a defibrillation port 410, such as a socket in the wearable housing 401. The defibrillation port 410 may include nodes 414 and 418. One or more electrodes 404 and 408 may be plugged in to the defibrillation port 410 facilitating an electrical contact with nodes 414 and 418, respectively. It may also be possible that the electrodes 404 and 408 may be connected continuously to the defibrillation port 410, etc. The defibrillation port 410 may be capable of retracting the electrodes 404 and 408, as described herein. As previously described, some, any, all, or any combination thereof of the components/modules illustrated in FIGS. 1-3 may be included in the WD 400 and may provide the various functionalities and/or any combination of functionalities described herein.

If the WD 400 may include external communication capabilities (e.g., TX/RX 220, and 320 of FIGS. 2-3), the WD 400 may also include an ECG port 419 in the WH 401, for receiving ECG leads 409. The ECG leads 409 may facilitate sensing of an ECG signal (e.g., a 12-lead signal or from a different number of lead signals). Moreover, external communication capabilities may include wireless communication capabilities and/or GPS capabilities, and the other component 425 may be configured to facilitate wireless communication and/or GPS location services.

The WD 400 also may include a measurement circuit 420. The measurement circuit 420 may receive physiological signals from the ECG port 419, and also from other ports, if provided. The measurement circuit 420 may render detected physiological signals and their corresponding information. The information may be in the form of data, or other signals, etc.

If the WD 400 may be configured as an AED type device as but one non-limiting example, ECG port 419 may not be present. The measurement circuit 420 may obtain physiological signals through the nodes 414 and 418 instead, when the electrodes 404 and 408 are attached to the person in need of the AED device. In other examples, the other component 525 may include a pulse sensor (pulse sensor 104, 204, and 304 of FIGS. 1-3) to facilitate at least some of the described functionalities.

The WD 400 may also include a processor 430 such as those described with respect to FIGS. 1-3 (e.g., processor 102, 202, and 302). The processor 430 may be implemented in a wide variety of manners for causing actions and operations to be performed and described herein. Some examples may include digital and/or analog processors such as microprocessors and digital-signal processors (DSPs), controllers such as microcontrollers, software running in a machine environment, programmable circuits such as Field Programmable Gate Arrays (FPGAs), Field-Programmable Analog Arrays (FPAAs), Programmable Logic Devices (PLDs), Application Specific Integrated Circuits (ASICs), and so on or any combination thereof.

The processor 430 may include a number of modules. One example module may be a detection module 432, which may detect outputs from the measurement circuit 420. The detection module 432 may include a VF detector. Accordingly, a person's detected ECG and/or pulse may be utilized to help determine whether the person is experiencing VF.

In another example, advice module 434 may provide advice based, at least in part, on outputs of the detection module 432. The advice module 434 may include an algorithm such as, but not limited to, Shock Advisory Algorithm, implement decision rules, and so on. For example, the advice may be to shock, to not shock, to administer other forms of therapy, and so forth. If the advice is to shock, some defibrillator examples may report the advice to the user and prompt them to do it. In other examples, the WD 400 may execute the advice by administering the shock. If the advice is to administer CPR, the WD 400 may further issue prompts for administrating CPR, and so forth.

The processor 430 may include additional modules, such as module 436 for various functions as described herein. For example, the other module 436 may be a module for facilitating at least some of the capabilities described herein. In another example, the other module 436 may be a module for facilitating measuring blood pressure. In yet another example, the other module 436 may be a module for facilitating detection and/or sensing motion (e.g., motion sensor 218 as described with respect to FIG. 2). For example, the WD 400 may include CPR training capabilities, as at least described herein.

In an example, the WD 400 may include a memory 438 (e.g., storage medium 110, 210, and 310 shown in FIGS. 1-3), which may work together with the processor 430. The memory 438 may be implemented in a wide variety of manners. For example, the memory 438 may be implemented such as, but not limited to, nonvolatile memories (NVM), read-only memories (ROM), random access memories (RAM), and so forth or any combination thereof. The memory 438 may include programs for the processor 430, and so on. The programs may include operational programs executed by the processor 430 and may also include protocols and methodologies so that decisions may be made by advice module 434. Additionally, the memory 438 may store various prompts for the user 480, etc. Moreover, the memory 438 may store a wide variety of information (i.e., data) such as, but not limited to information regarding a WHUM (e.g., WHUM 108, 208, and 308 of FIGS. 1-3).

The WD 400 may also include a power source 440 such as, but not limited to, those shown in FIG. 3 (power supply 324). In order to facilitate wear ability of WD device 400, the power source 440 may include a battery type device. A battery type device may be implemented as a battery pack, which may be rechargeable or non-rechargeable. At times, a combination of rechargeable and non-rechargeable battery packs may be utilized. Examples of power source 440 may include AC power override, where AC power may be available, and so on. In some examples, the processor 430 may control the power source 440. As previously described, in one example, the power source 440 may provide power to the various components of FIGS. 1-3.

Additionally, the WD 400 may include an energy storage module 450. The energy storage module 450 may be configured to store some electrical energy (e.g., when preparing for sudden discharge to administer a shock). The energy storage module 450 may be charged from the power source 440 to an appropriate level of energy, as may be controlled by the processor 430. In some implementations, the energy storage module 450 may include one or more capacitors 452, and the like.

The WD 400 may include a discharge circuit 455. The discharge circuit 455 may be controlled to facilitate discharging of the energy stored in energy storage module 450 to the nodes 414 and 418, and also to electrodes 404 and 408. The discharge circuit 455 may include one or more switches 457. The one or more switches 457 may be configured in a number of manners such as, but not limited to, an H-bridge, and so forth.

The WD 400 may further include a user interface 470 such as, but not limited to, those described with respect to FIGS. 1-3 (visual indicator device 106, 206, and 306) for the user 480. The user interface 470 may be implemented in a variety of manners as previously described. For example, the user interface 470 may include a display screen capable of displaying what is detected and measured, provide visual feedback to the user 480 for their resuscitation attempts, and so forth. The user interface 470 may also include an audio output such as, but not limited to, a speaker to issue audio prompts, etc., as previously described with respect to FIG. 2. The user interface 470 may additionally include various control devices such as, but not limited to, pushbuttons, keyboards, switches, track pads, touchscreen, and so forth. Additionally, the discharge circuit 455 may be controlled by the processor 430 or directly by the user 480 via the user interface 470, and so forth.

Additionally, the WD 400 may include other components. For example, a communication module 490 may be provided for communicating with other machines and/or other services such as, but not limited to, those communication as previously described with respect to FIGS. 2-3 (TX/RX 220 and 320 shown in FIGS. 2-3). Such communication may be performed wirelessly, or via wire, or by infrared communication, near field communication (NFC), Bluetooth, Wi-Fi, and so forth. Accordingly, information may be communicated, such as person data, incident information, therapy attempted, CPR performance, ECG information, and so forth.

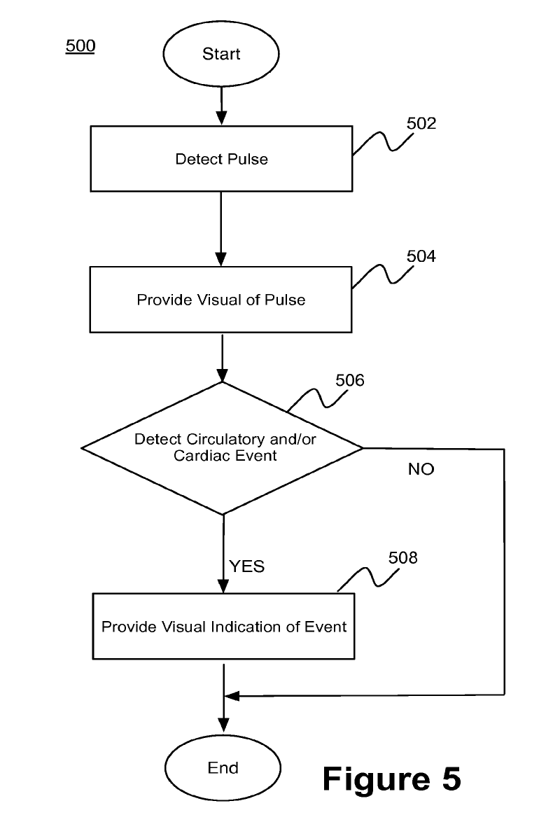

FIG. 5 illustrate an operational flow for a wearable healthcare device, arranged in accordance with at least some embodiments described herein. In some portions of the description, illustrative implementations of the method are described with reference to the elements, components, and/or any combination thereof of apparatuses depicted in FIGS. 1-4. However, the described embodiments are not limited to these depictions. More specifically, some elements depicted in FIGS. 1-4 may be omitted from some implementations of the methods details herein. Furthermore, other elements not depicted in FIGS. 1-4 may be used to implement example methods detailed herein.

Additionally, FIG. 5 employs block diagrams to illustrate the example methods detailed therein. These block diagrams may set out various functional block or actions that may be described as processing steps, functional operations, events and/or acts, etc., and may be performed by hardware, software, and/or firmware. Numerous alternatives to the functional blocks detailed may be practiced in various implementations. For example, intervening actions not shown in the figures and/or additional actions not shown in the figures may be employed and/or some of the actions shown in one figure may be operated using techniques discussed with respect to another figure. Additionally, in some examples, the actions shown in these figures may be operated using parallel processing techniques. The above described, and other not described, rearrangements, substitutions, changes, modifications, etc., may be made without departing from the scope of the claimed subject matter.

In some examples, operational flow 500 may be employed as part of an wearable healthcare device. Beginning at block 502 (“Detect Pulse”), a WD may detect the pulse of a wearer.

Continuing from block 502 to block 504 (“Provide Visual of Pulse”), a visual indicator as described may be configured to provide various information including the pulse of the wearer.

Continuing from block 504 to block 506 (“Detect a Circulatory and/or Cardiac Related Event”), the WHUM receive an indication of a circulatory and/or cardiac related event such as, but not limited to, VF, as previously described.

Continuing from block 508 to block 508 (“Provide Visual Indication of the Event”), the WHUM may upon receiving the indication of a circulatory and/or cardiac related event, provide a visual indication of the event such as, but not limited to, blinking lights, message on a display, and various other implementations and/or methodologies described, and/or any combination thereof.

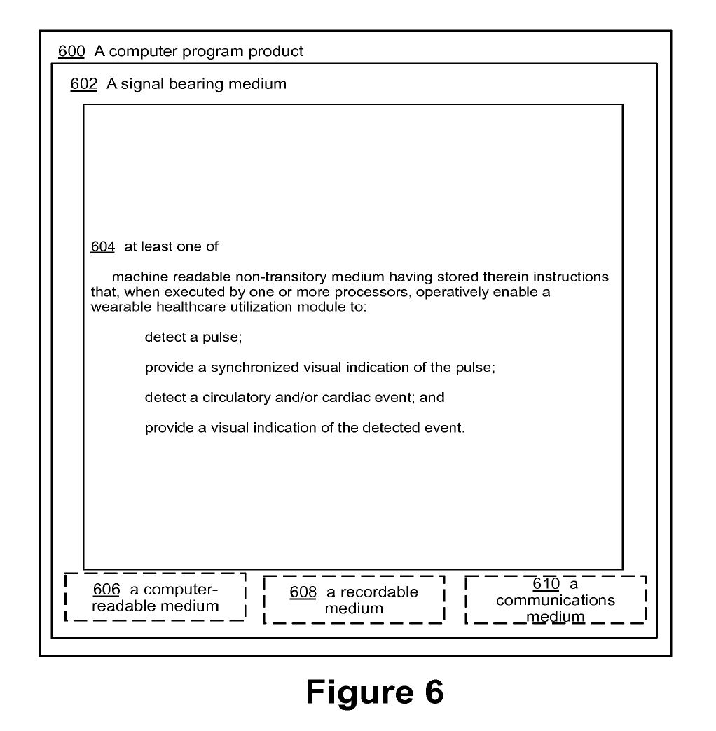

In general, the operational flow described with respect to FIG. 5 and elsewhere herein may be implemented as a computer program product, executable on any suitable computing system, or the like. For example, a computer program product for facilitating at least the various examples described herein may be provided. Example computer program products may be described with respect to FIG. 6 and elsewhere herein.

FIG. 6 illustrates an example computer program product 600, arranged in accordance with at least some embodiments described herein. Computer program product 600 may include machine readable non-transitory medium having stored therein instructions that, when executed, cause the machine to visually indicate a pulse of wearer and detect a health related event, according to the processes and methods discussed herein. Computer program product 600 may include a signal bearing medium 602. Signal bearing medium 602 may include one or more machine-readable instructions 604 which, when executed by one or more processors, may operatively enable a computing device to provide the functionality described herein. In various examples, the devices discussed herein may use some or all of the machine-readable instructions.

In some examples, the machine readable instructions 604 may include instructions to detect a pulse. In some examples, the machine readable instructions 904 may include instructions to synchronously visually indicate the pulse. In some examples, the machine readable instructions 604 may include instructions to detect a circulatory and/or a cardiac event provide a visual indication of the detected event, and upon detecting the indication of a circulatory and/or cardiac related event, the machine readable instructions may provide a visual indication of the event such as, but not limited to, blinking lights, message on a display, and various other implementations and/or methodologies described, and/or any combination thereof.

In some implementations, signal bearing medium 602 may encompass a computer-readable medium 606, such as, but not limited to, a hard disk drive, a Compact Disc (CD), a Digital Versatile Disk (DVD), a Universal Serial Bus (USB) drive, a digital tape, memory, etc. In some implementations, the signal bearing medium 602 may encompass a recordable medium 608, such as, but not limited to, memory, read/write (R/W) CDs, R/W DVDs, etc. In some implementations, the signal bearing medium 602 may encompass a communications medium 610, such as, but not limited to, a digital and/or an analog communication medium (e.g., a fiber optic cable, a waveguide, a wired communication link, a wireless communication link, etc.). In some examples, the signal bearing medium 602 may encompass a machine readable non-transitory medium.

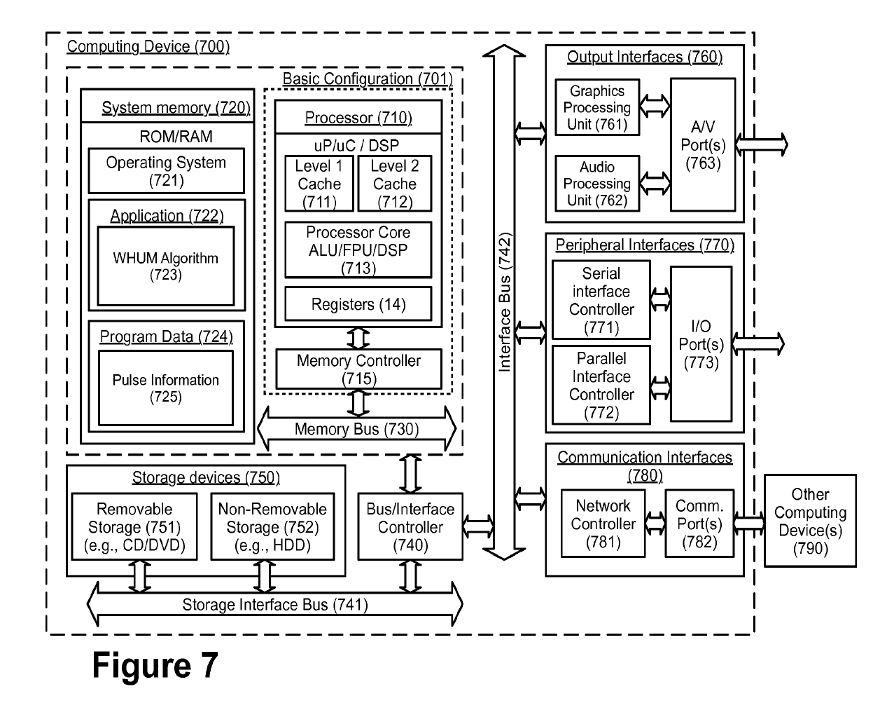

In general, the methods described with respect to FIG. 6, and elsewhere herein may be implemented in any suitable computing system. Example systems may be described with respect to FIG. 7 and elsewhere herein. In general, the system may be configured to facilitate various methodologies and/or implementations as at least described herein.

FIG. 7 is a block diagram illustrating an example computing device 700, such as might be embodied by a person skilled in the art, which is arranged in accordance with at least some embodiments of the present disclosure. In one example configuration 701, computing device 700 may include one or more processors 710 and system memory 720. A memory bus 730 may be used for communicating between the processor 710 and the system memory 720.

Depending on the desired configuration, processor 710 may be of any type including but not limited to a microprocessor (μP), a microcontroller (μC), a digital signal processor (DSP), or any combination thereof. Processor 710 may include one or more levels of caching, such as a level one cache 711 and a level two cache 712, a processor core 713, and registers 714. The processor core 713 may include an arithmetic logic unit (ALU), a floating point unit (FPU), a digital signal processing core (DSP Core), or any combination thereof. A memory controller 715 may also be used with the processor 710, or in some implementations the memory controller 715 may be an internal part of the processor 710.

Depending on the desired configuration, the system memory 720 may be of any type including but not limited to volatile memory (such as RAM), non-volatile memory (such as ROM, flash memory, etc.) or any combination thereof. System memory 720 may include an operating system 721, one or more applications 722, and program data 724. Application 722 may include indication of a wearable healthcare utilization (WHUM) algorithm 723 that is arranged to perform the functions as described herein including the functional blocks and/or actions described. Program Data 724 may include, among a wide variety of information described, pulse information 725 for use with WHUM algorithm 723. In some example embodiments, application 722 may be arranged to operate with program data 724 on an operating system 721 such that implementations of visual indication of a pulse may be provided as described herein. For example, apparatus described in the present disclosure may comprise all or a portion of computing device 700 and be capable of performing all or a portion of application 722 such that implementations of a wearable healthcare apparatus may be provided as described herein. This described basic configuration is illustrated in FIG. 7 by those components within dashed line 701.

Computing device 700 may have additional features or functionality, and additional interfaces to facilitate communications between the basic configurations 701 and any required devices and interfaces. For example, a bus/interface controller 740 may be used to facilitate communications between the basic configuration 701 and one or more data storage devices 750 via a storage interface bus 741. The data storage devices 750 may be removable storage devices 751, non-removable storage devices 752, or a combination thereof. Examples of removable storage and non-removable storage devices include magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include volatile and nonvolatile, removable and non-removable media implemented in any method or technology for storage of information, such as computer readable instructions, data structures, program modules, or other data.

System memory 720, removable storage 751 and non-removable storage 752 are all examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 700. Any such computer storage media may be part of device 700.

Computing device 700 may also include an interface bus 742 for facilitating communication from various interface devices (e.g., output interfaces, peripheral interfaces, and communication interfaces) to the basic configuration 701 via the bus/interface controller 740. Example output interfaces 760 may include a graphics processing unit 761 and an audio processing unit 762, which may be configured to communicate to various external devices such as a display or speakers via one or more A/V ports 763. Example peripheral interfaces 760 may include a serial interface controller 771 or a parallel interface controller 772, which may be configured to communicate with external devices such as input devices (e.g., keyboard, mouse, pen, voice input device, touch input device, etc.) or other peripheral devices (e.g., printer, scanner, etc.) via one or more I/O ports 773. An example communication interface 780 includes a network controller 781, which may be arranged to facilitate communications with one or more other computing devices 790 over a network communication via one or more communication ports 782. A communication connection is one example of a communication media. Communication media may typically be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), infrared (IR) and other wireless media. The term computer readable media as used herein may include both storage media and communication media.

Computing device 700 may be implemented as a portion of a wearable healthcare device, a small-form factor portable (or mobile) electronic device such as a cell phone, a personal data assistant (PDA), a tablet type device, a personal media player device, a wireless web-watch device, a personal headset device, an application specific device, or a hybrid device that includes any of the above functions. Computing device 700 may also be implemented as a personal computer including both laptop computer and non-laptop computer configurations. In addition, computing device 700 may be implemented as part of a wireless base station or other wireless system or device.

Some portions of the foregoing detailed description are presented in terms of algorithms or symbolic representations of operations on data bits or binary digital signals stored within a computing system memory, such as a computer memory. These algorithmic descriptions or representations are examples of techniques used by those of ordinary skill in the data processing arts to convey the substance of their work to others skilled in the art. An algorithm is here, and generally, considered to be a self-consistent sequence of operations or similar processing leading to a desired result. In this context, operations or processing involve physical manipulation of physical quantities. Typically, although not necessarily, such quantities may take the form of electrical or magnetic signals capable of being stored, transferred, combined, compared or otherwise manipulated. It has proven convenient at times, principally for reasons of common usage, to refer to such signals as bits, data, values, elements, symbols, characters, terms, numbers, numerals or the like. It should be understood, however, that all of these and similar terms are to be associated with appropriate physical quantities and are merely convenient labels. Unless specifically stated otherwise, as apparent from the following discussion, it is appreciated that throughout this specification discussion utilizing terms such as “processing,” “computing,” “calculating,” “determining” or the like refer to actions or processes of a computing device that manipulates or transforms data represented as physical electronic or magnetic quantities within memories, registers, or other information storage devices, transmission devices, or display devices of the computing device.

Claimed subject matter is not limited in scope to the particular implementations described herein. For example, some implementations may be in hardware, such as those employed to operate on a device or combination of devices, for example, whereas other implementations may be in software and/or firmware. Likewise, although claimed subject matter is not limited in scope in this respect, some implementations may include one or more articles, such as a signal bearing medium, a storage medium and/or storage media. This storage media, such as CD-ROMs, computer disks, flash memory, or the like, for example, may have instructions stored thereon that, when executed by a computing device such as a computing system, computing platform, or other system, for example, may result in execution of a processor in accordance with claimed subject matter, such as one of the implementations previously described, for example. As one possibility, a computing device may include one or more processing units or processors, one or more input/output devices, such as a display, a keyboard and/or a mouse, and one or more memories, such as static random access memory, dynamic random access memory, flash memory, and/or a hard drive.

There is little distinction left between hardware and software implementations of aspects of systems; the use of hardware or software is generally (but not always, in that in certain contexts the choice between hardware and software can become significant) a design choice representing cost vs. efficiency tradeoffs. There are various vehicles by which processes and/or systems and/or other technologies described herein can be affected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware would be well within the skill of one of skilled in the art in light of this disclosure. In addition, those skilled in the art will appreciate that the mechanisms of the subject matter described herein are capable of being distributed as a product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a flexible disk, a hard disk drive (HDD), a Compact Disc (CD), a Digital Versatile Disk (DVD), a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Reference in the specification to “an implementation,” “one implementation,” “some implementations,” or “other implementations” may mean that a particular feature, structure, or characteristic described in connection with one or more implementations may be included in at least some implementations, but not necessarily in all implementations. The various appearances of “an implementation,” “one implementation,” or “some implementations” in the preceding description are not necessarily all referring to the same implementations.

While certain exemplary techniques have been described and shown herein using various methods and systems, it should be understood by those skilled in the art that various other modifications may be made, and equivalents may be substituted, without departing from claimed subject matter. Additionally, many modifications may be made to adapt a particular situation to the teachings of claimed subject matter without departing from the central concept described herein. Therefore, it is intended that claimed subject matter not be limited to the particular examples disclosed, but that such claimed subject matter also may include all implementations falling within the scope of the appended claims, and equivalents thereof.

What is claimed:

1. A wearable healthcare apparatus comprising:
a processor;
a wearable healthcare utilization module (WHUM) communicatively coupled to the processor;
a pulse sensor communicatively coupled to the processor and configured to sense an indication of a pulse of a wearer of the wearable healthcare apparatus; and
a visual indicator device configured to synchronously visually indicate the pulse of the wearer of the wearable healthcare apparatus.

2. The wearable healthcare apparatus of claim 1, wherein the pulse sensor comprises a peripheral capillary oxygen saturation device.

3. The wearable healthcare apparatus of claim 1 further comprising:
a digital to analog converter (DAC) communicatively coupled to the processor;
and a speaker communicatively coupled to the DAC.

4. The wearable healthcare apparatus of claim 1, wherein the visual indicator device comprises a liquid crystal display (LCD) device.

5. The wearable healthcare apparatus of claim 1, wherein the visual indicator device comprises a plurality of light emitting diodes (LEDs) configured to indicate an intensity of the pulse of the wearer.

6. The wearable healthcare apparatus of claim 1 further comprising:
a band configured to go around an arm of the wearer and tighten and loosen around the arm under the control of the processor.

7. The wearable healthcare apparatus of claim 1 further comprising:
a motion sensor communicatively coupled to the processor; and
a wireless communication device communicatively coupled to the processor.

8. The wearable healthcare apparatus of claim 7, wherein the motion sensor comprises a motion sensor configured to sense a range of motion related to cardiopulmonary resuscitation (CPR) activity of the wearer.

9. The wearable healthcare apparatus of claim 1 further comprising: a storage device communicatively coupled to the processor.

10. The wearable healthcare apparatus of claim 9, wherein the storage device comprises a storage device configured to store electrocardiography (ECG) data.
